(19) United States
(12) Patent Application Publication
(10) Pub. No.: US 2026/0260718 A1
(43) Pub. Date: Sep. 3, 2026

(54) INFORMATION PROCESSING SYSTEM, INFORMATION PROCESSING METHOD, AND INFORMATION PROCESSING PROGRAM

(71) Applicant: QUEMIX INC., Tokyo (JP)

(72) Inventors: Yuichiro MATSUSHITA, Tokyo (JP); Hung Ba TRAN, Tokyo (JP)

(21) Appl. No.: 18/878,484

(22) PCT Filed: Jun. 30, 2023
(86) PCT No.: PCT/JP2023/024400

§ 371 Date: Dec. 23, 2024

(30) Foreign Application Priority Data
Jul. 12, 2022 (JP) 2022-112076

(51) Int. Cl. G16C 60/00; G06F 30/20

(52) U.S. Cl. CPC G16C 60/00; G06F 30/20

(57) ABSTRACT

An information processing system includes at least one processor to execute a program to perform: an acquisition step of acquiring first estimated data related to a physical property of a material, which is calculated by predetermined physical property simulation based on a model of the material having a ferroic order phase, and measured data obtained by measurement of the material; a data assimilation processing step of, when the measured data includes a measured reference value, executing first data assimilation processing on the coupling coefficient by multiplying a ratio of the measured reference value to an estimated reference value with the coupling coefficient included in the acquired first estimated data according to a degree which represents dependence of the estimated reference value on the coupling coefficient; and an output step of outputting the coupling coefficient, on which the first data assimilation processing is executed, as second estimated data.

### CROSS REFERENCE TO RELATED APPLICATIONS

This application is a 371 U.S. National Phase of International Application No. PCT/JP2023/024400, filed on Jun. 30, 2023, which claims priority to Japanese Patent Application No. 2022-112076, filed Jul. 12, 2022. The entire disclosures of the above applications are incorporated herein by reference.

### BACKGROUND

The present disclosure relates to an information processing system, an information processing method and an information processing program.

### RELATED ART

As a conventional art, JP 2021-33964 A discloses a saturation magnetization prediction method and a saturation magnetization prediction simulation program, which can easily calculate saturation magnetization of a magnetic phase at a finite temperature.

The magnetization prediction method includes: a first step of calculating saturation magnetization at an absolute zero point and a Curie temperature by substituting measured data of saturation magnetization at a finite temperature into the Kuzmin formula; a second step of executing data assimilation respectively on the saturation magnetization at the absolute zero point and the Curie temperature which are obtained in the first step and saturation magnetization at the absolute zero point and a Curie temperature which are obtained by first-principles calculation so as to calculate a prediction model formula, which represents the saturation magnetization at the absolute zero point and the Curie temperature respectively by a function formula on existing proportions of elements that compose a single magnetic phase, by machine learning; and a third step of calculating the saturation magnetization at the finite temperature by applying the prediction model created in the second step to the Kuzmin formula.

By the way, estimated data of a material having a ferroic order phase by physical property simulation may be different from actual measured data. Factors causing such a difference between the estimated data and the measured data are varied, which include a factor resulted from a measured sample such as purity and a shape of the material, a factor resulted from approximation in the physical property simulation, and the like, and may also be varied depending on a state, measurement conditions and the like of the measured sample. Therefore, there is still room for improvement in the art to incorporate the factors, which cause such a difference between the estimated data and the measured data, into the estimated data.

### SUMMARY

According to one aspect of the present disclosure, an information processing system is provided. This information processing system includes at least one processor that can execute a program to perform each of following steps. An acquisition step acquires: first estimated data related to physical properties of a material which is calculated by predetermined physical property simulation based on a model of the material having a ferroic order phase; and measured data obtained by measurement on the material. Herein, the first estimated data includes: temperature dependence of an order parameter in the ferroic order phase; and a coupling coefficient representing magnitude of interaction between sites of the material, which contributes to formation of the ferroic order phase. In a data assimilation processing step, when the measured data includes a measured reference value, first data assimilation processing is executed on the said coupling coefficient by multiplying: a ratio of the measured reference value to an estimated reference value; with the coupling coefficient included in the acquired first estimated data, according to a degree which represents dependence of the estimated reference value on the coupling coefficient. Herein, the measured reference value includes at least one of: a phase transition temperature representing phase transition from the ferroic order phase which is caused due to the order parameter becoming zero; and a saturation value which is a value of the order parameter corresponding to a saturated state of the ferroic order phase at an absolute zero point. The estimated reference value is a value corresponding to the measured reference value among the phase transition temperature and the saturation value which are included in the acquired first estimated data. An output step outputs the coupling coefficient, on which the first data assimilation processing is executed, as second estimated data.

According to this configuration, the first estimated data includes information that is related to ideal physical properties of the material as a target to be measured. The measured data includes information, which is intrinsic to the material as the target to be measured, such as quality of the material and measurement conditions. Therefore, the second estimated data, which is calculated from the first estimated data and the measured data, includes the information intrinsic to the material that is reflected on the ideal physical properties of the material.

Herein, the coupling coefficient represents strength of the interaction between the sites forming the ferroic order phase. Therefore, the coupling coefficient is an important factor for specifying characteristics of the ferroic order phase such as a physical property value generated by the ferroic order phase and a spatial property for domain formation and the like.

Accordingly, by enhancing accuracy of the coupling coefficient by the above-described data assimilation, a deviation between a result of the physical property simulation on the ferroic order phase and a measurement result of the material can be suppressed.

### BRIEF DESCRIPTION OF THE DRAWINGS

FIG. 1 is a diagram that represents an information processing system 1.

FIG. 2 is a block diagram that illustrates a hardware configuration of an information processing apparatus 2.

FIG. 3 is a block diagram that illustrates a hardware configuration of a user terminal 3.

FIG. 4 is a view that illustrates an example of a functional unit included in a processor 23.

FIG. 5 is a flowchart that represents an example of a flow of information processing executed by the information processing system 1.

FIG. 6 is a flowchart that represents a flow of data assimilation processing.

FIG. 7 is a flowchart that represents details of processing in Step S100.

FIG. 8 is a view that illustrates a change in temperature dependence of spontaneous magnetization M due to data assimilation in Step S103.

FIG. 9 is a view that illustrates a change in temperature dependence of spontaneous magnetization M due to data assimilation in Step S110.

FIG. 10 is a flowchart that represents details of processing in Step S200.

FIG. 11 is a view that illustrates a change in temperature dependence of magnetic anisotropy energy K due to data assimilation in Step S202.

FIG. 12 is a view that illustrates a change in temperature dependence K2 of second magnetic anisotropy energy K due to correction in Step S204.

FIG. 13 is a flowchart that represents details of processing in Step S300.

FIG. 14 is a flowchart that represents details of processing in Step S305.

### DETAILED DESCRIPTION

Hereinafter, embodiments of the present disclosure will be described with reference to the drawings. Various features shown in the following embodiment can be combined with each other.

By the way, the program for realizing software used in this embodiment may be provided as a non-transitory computer-readable medium, may be provided to be downloadable from an external server, or may be provided so that the program can be started on an external computer to realize its functions on a client terminal (so-called cloud computing).

In this embodiment, the term “part” in the present embodiment may include, for example, hardware resources implemented by circuits in a broad sense, together with information processing of software which can be specifically realized by those hardware resources. In addition, although various types of information are handled in this embodiment, such information can be represented by physical values of signal values representing, for example, voltage and current, high and low signal values as a binary bit array consisting of 0 or 1, or quantum superposition (so-called quantum bits), and communication and computation can be executed on a circuit in a broad sense.

A circuit in a broad sense means a circuit realized by combining a circuit, circuitry, a processor, a memory and the like in at least appropriate combination. That is, such circuits include an application specific integrated circuit (ASIC), a programmable logic device (e.g., a simple programmable logic device (SPLD), a complex programmable logic device (CPLD), etc.), and an integrated circuit (IC), a field programmable gate array (FPGA) and the like.

### 1. Hardware Configuration

This section will provides explanation of a hardware configuration.

### <Information Processing System 1>

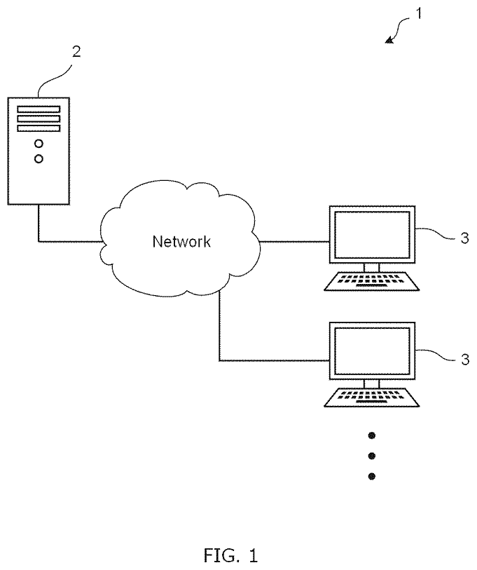

FIG. 1 is a diagram that represents an information processing system 1. The information processing system 1 includes an information processing apparatus 2 and a user terminal 3. The information processing apparatus 2 and the user terminal 3 are configured to be able to communicate via a telecommunication line. In one embodiment, the information processing system 1 is composed mainly of one or more devices or components. If, for example, the information processing system 1 is composed only of the information processing apparatus 2, the information processing system 1 can be the information processing apparatus 2. Hereinafter, explanation of these components will be provided.

### <Information Processing Apparatus 2>

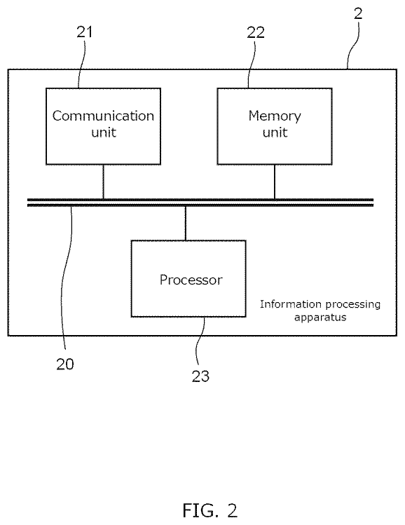

FIG. 2 is a block diagram that illustrates a hardware configuration of the information processing apparatus 2. The information processing apparatus 2 includes a communication unit 21, a memory unit 22 and a processor 23, and these components are electrically connected via a communication bus 20 inside the information processing apparatus 2. Each of the component will be further described below.

The communication unit 21 is preferably a wire communication means such as USB, IEEE1394, Thunderbolt (registered trademark), wired LAN network communication, etc., but may also include wireless LAN network communication, mobile communication such as 3G/LTE/5G, Bluetooth (registered trademark) communication, and the like as necessary. More preferably, integration of these plural communication means is used. That is, the information processing apparatus 2 may communicate various information from outside via the communication unit 21 and a network.

The memory unit 22 stores various information defined by the above description. This is, for example, the memory unit 22 can store such information as a storage device such as a solid state drive (SSD) that stores various programs, etc. related to the information processing apparatus 2, which are executed by the processor 23, or as a memory such as a random access memory (RAM) that stores temporarily necessary information (arguments, arrays, etc.) for program calculations. The memory unit 22 stores various programs and variables related to the information processing apparatus 2, which are executed by the processor 23.

The processor 23 processes and controls overall operations related to the information processing apparatus 2. The processor 23 is, for example, an unshown central processing unit (CPU). The processor 23 reads out a predetermined program stored in the memory unit 22 so as to realize various functions related to the information processing apparatus 2. That is, the information processing by software stored in the memory unit 22 is specifically realized by the processor 23 as an example of hardware, and can be executed by each functional unit included in the processor 23. They will be described in more detail in a next section. Incidentally, the processor 23 is not limited to be single, but may be implemented so as to include a plurality of the processors 23 for each function. Also, the processor 23 may be a combination of the structures described above.

### <User Terminal 3>

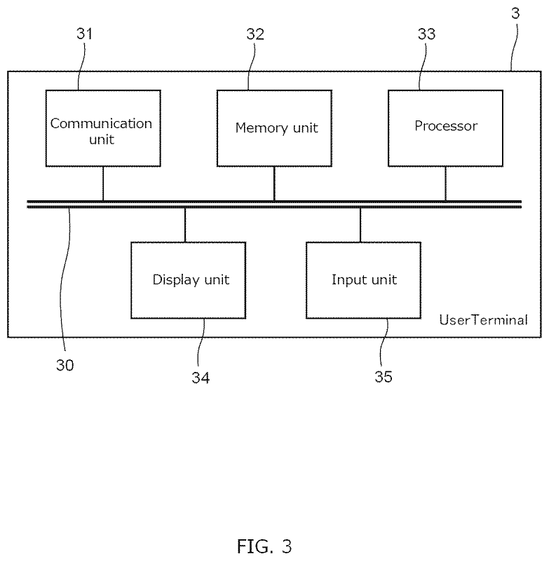

FIG. 3 is a block diagram that illustrates a hardware configuration of the user terminal 3. The user terminal 3 includes a communication unit 31, a memory unit 32, a processor 33, a display unit 34 and an input unit 35, and these components are electrically connected via a communication bus 30 inside the user terminal 3. Explanation of the communication unit 31, the memory unit 32 and the processor 33 is the same as that of the respective units in the information processing apparatus 2, and is therefore omitted.

The display unit 34 may be included in a housing of the user terminal 3, or may be attached thereto externally. The display unit 34 displays a screen of a graphical user interface (GUI) that can be operated by a user. This displaying is preferably performed by a display device, for example, a CRT display, a liquid crystal display, an organic EL display, a plasma display and the like, depending on a type of the user terminal 3.

The input unit 35 may be included in the housing of the user terminal 3, or may be attached thereto externally. For example, the input unit 35 may be integrated with the display unit 34 and implemented as a touch panel. If the input unit 35 is implemented as a touch panel, the user can input by tap operations, swipe operations and the like. Needless to say, a switch button, a mouse, a QWERTY keyboard or the like may be employed instead of a touch panel. Accordingly, the input unit 35 accepts an operation input performed by the user. The input is transferred as an instruction signal via the communication bus 30 to the processor 33, and the processor 33 can execute predetermined control or arithmetic operations as necessary.

### 2. Functional Configuration of Information Processing Apparatus 2

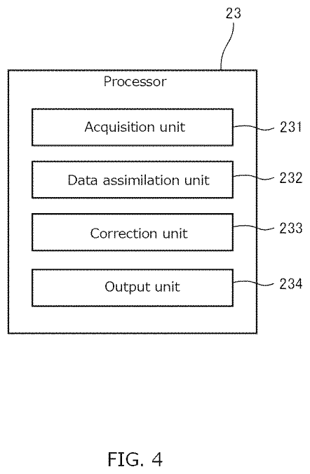

FIG. 4 is a view that illustrates an example of a functional unit included in the processor 23. As shown in FIG. 4, the processor 23 includes an acquisition unit 231, a data assimilation unit 232, a correction unit 233 and an output unit 234.

The acquisition unit 231 is configured to be able to acquire information from the user terminal 3 or other devices. The acquisition unit 231 is configured to be able to acquire, for example, first estimated data that is a result of calculation by predetermined physical property simulation, measured data obtained by measurement on the material, a relational expression that represents a function formula of a physical property as the target and a field, and the like. Details thereof will be described below.

The acquisition unit 231 is configured to be able to acquire various information by: reading out the various information stored in a storage area, which is at least a part of the memory unit 22; and writing the readout information into a working area, which is at least a part of the memory unit 22. The storage area is, for example, an area in the memory unit 22, which is implemented as a storage device such as an SSD. The working area is, for example, an area which is implemented as a memory such as an RAM.

The data assimilation unit 232 is configured to execute data assimilation on the first estimated data according to the measured data acquired by the acquisition unit 231. The data assimilation unit 232 is configured to be able to generate second estimated data by executing the said data assimilation. The second estimated data can be considered also as the first estimated data on which the data assimilation is executed.

The correction unit 233 is configured to be able to correct the acquisition results acquired by the acquisition unit 231 and the results of the data assimilation processing executed by the data assimilation unit 232, by using various parameters.

The output unit 234 is configured to be able to output various information such as the first estimated data and the second estimated data. The said information can be shown to the user via the display unit 34 of the user terminal 3 or other devices. In such a case, for example, the output unit 234 controls the display unit 34 of the user terminal 3 to display visual information such as screens, images including still or moving images, icons, messages and the like. The output unit 234 may generate only rendering information for displaying the visual information on the user terminal 3. Incidentally, the output unit 234 may show the output information to the user not via the user terminal 3 or any other device users.

### 3. Regarding Information Processing

This section will provide explanation of the information processing to be executed by the information processing system 1. The said information processing uses, for example, a result of simulation on a model of a material which has a ferroic order phase so as to be used for simulating a dynamic property of the ferroic order phase. Hereinafter, an example of the information processing on the material having a ferromagnetic phase as the ferroic order phase (ferromagnetic material) will be described. The ferromagnetic material is, for example, an iron magnet such as Fe, Fe₃O₄, FePt, Ni—Zn, ferrite and the like. Incidentally, the ferromagnetic material is not limited to the above and is arbitrary, and may be an inorganic compound magnet such as a Co magnet, an Ni magnet and a Nd magnet, or an organic magnet.

### 3.1. Flow of Information Processing

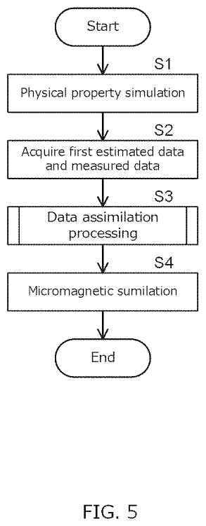

FIG. 5 is a flowchart that represents an example of a flow of the information processing executed by the information processing system 1. Incidentally, the said information processing may include arbitrary exception processing, which is not shown in the figure. The exception processing includes interruption of the said information processing and omission of each process. Selection or input performed in the said information processing may be based on operations by the user, or may be automatic not by the user's operations.

### [Step S1]

Firstly, in Step S1, the acquisition unit 231 acquires a model of a material having a ferromagnetic phase, and the processor 23 executes predetermined physical property simulation based on the acquired model. Thereby, the output unit 234 outputs the first estimated data related to the physical property of the ferromagnetic material. The first estimated data includes temperature dependence of an order parameter in the ferroic order phase and a coupling coefficient. The first estimated data may further include temperature dependence of anisotropy energy, temperature dependence of an exchange stiffness constant A, temperature dependence of a damping constant α and the like.

### <Temperature Dependence of Order Parameter>

The temperature dependence of the order parameter in the ferroic order phase may include a saturation value and a phase transition temperature. The saturation value is a value of the order parameter corresponding to a saturated state of the ferroic order phase at the absolute zero point. The phase transition temperature represents phase transition from the ferroic order phase caused due to the order parameter becoming zero. In this embodiment, since the ferroic order phase is the ferromagnetic phase, the order parameter is spontaneous magnetization M of the material. The saturated state is a state in which the material as the target has a domain structure that exhibits a substantially single ferroic order. The saturation value is saturation magnetization of the material, in particular, saturation magnetization M0 at the absolute zero point. The phase transition temperature is a Curie temperature Tc that corresponds to phase transition from the ferromagnetic phase to paramagnetic phase. That is, the temperature dependence of the order parameter in the ferroic order phase is temperature dependence of the spontaneous magnetization M in the ferromagnetic phase. The spontaneous magnetization M of the present embodiment has the temperature dependence which is, for example, decreased from the saturation magnetization M0 according to increase of the temperature, and becomes zero at the Curie temperature Tc. Hereinafter, for convenience of explanation, the temperature dependence of the spontaneous magnetization M included in the first estimated data is denoted by temperature dependence M1 of the first spontaneous magnetization, and the Curie temperature Tc included in the first estimated data is denoted by a first estimated Curie temperature Tc1.

### <Coupling Coefficient>

The coupling coefficient represents the magnitude of the interaction between the sites of the material, which contributes to the formation of the ferroic order phase. The coupling coefficient of the present embodiment is a magnetic exchange coefficient Jij according to the fact that the ferroic order phase is the ferromagnetic phase. The magnetic exchange coefficient Jij represents the interaction between the sites. Specifically, the magnetic exchange coefficient Jij represents interaction between spins located at an i-th site and a j-th site in the material. The interaction between the spins may include exchange interaction between the spins, magnetic interaction between the spins and the like. The magnetic exchange coefficient Jij defines, for example, first Hamiltonian H1 that corresponds to the exchange energy between the spins.

$$H 1 = - {\sum }_{< ij >} {J}_{ij} {s}_{i} \cdot {s}_{j} \qquad [ Formula 1 ]$$

Incidentally, i and j are indices that represent the sites in the material. A sign of S_i denotes a spin operator of the i-th site. The spin operator S_i in the present embodiment is represented by a classical Heisenberg model of Si=(S_ix, S_iy, S_iz). Incidentally, the model representing such a spin system of the material is not limited to this model, but models such as an Ising model and an XY model can be set appropriately according to the system to be solved. For convenience of explanation, the magnetic exchange coefficient Jij included in the first estimated data is hereinafter denoted by a first estimated magnetic exchange coefficient Jij1.

### <Exchange Stiffness Constant A>

The exchange stiffness constant A is quantity that represents magnitude of a change in exchange energy per unit volume. The exchange stiffness constant A can be calculated from the magnetic exchange coefficient Jij. An exchange stiffness constant A0 at the absolute zero point is represented by a below-described total sum of the magnetic exchange coefficients Jij obtained by adopting, for example, mean field approximation.

$${A}_{0} = \frac{n}{2 a} {\sum }_{ij} {J}_{ij} \qquad [ Formula 2 ]$$

Herein, n denotes an atomic number included in a cell of the material as the target to be calculated, and a denotes a lattice constant of the cell.

In addition, in the mean field approximation, temperature dependence of the exchange stiffness constant A is represented as follows, by using the exchange stiffness constant A0, the saturation magnetization M0 and the temperature dependence of the spontaneous magnetization M at the absolute zero point.

$$A ( T ) = A 0 \times {( \frac{M ( T )}{M 0} )}^{2} \qquad [ Formula 3 ]$$

### <Anisotropy Energy>

The anisotropy energy represents magnitude of anisotropy of the order parameter in the ferroic order phase of the material. The anisotropy energy of the present embodiment is magnetic anisotropy energy K (MAE). The magnetic anisotropy energy K is varied according to a direction of the spin in the ferromagnetic material. The magnetic anisotropy energy K may include contribution by second Hamiltonian H2 resulted from uniaxial anisotropy of the spin, third Hamiltonian H3 resulted from symmetry of a crystal structure and the like. The third Hamiltonian H3 in the present embodiment is that of case where the material has a cubic crystal.

$$H 2 = - {\sum }_{i} {{k}_{u} ( {e}_{u} \cdot {S}_{i} )}^{2} \qquad [ Formula 4 ]$$

$$H 3 = - {\sum }_{i} {k}_{c} ( {S}_{ix}^{4} + {S}_{iy}^{4} + {S}_{iz}^{4} ) \qquad [ Formula 5 ]$$

Incidentally, μ is an index denoting any one of x, y and z directions that represent coordinates, and e_u denotes a unit vector in the direction that corresponds to μ. Signs of k_u and k_c are parameters that respectively represent extents of magnetic anisotropy, and are determined by, for example, kind of atoms, crystal structures, a distance between the sites, and the like. Hereinafter, for convenience of explanation, the temperature dependence of the magnetic anisotropy energy K included in the first estimated data is denoted simply by the temperature dependence K1 of the first estimated magnetic anisotropy energy.

### <Model of Material>

The model of the material includes, for example, Hamiltonian as the target, information related to the crystal structure of the material, an approximation method of the physical property of the material (a type, magnitude, a format of expression and the like of the interaction to be incorporated into the calculation) and the like. The Hamiltonian as the target is set appropriately according to a system to be focused on. For example, the Hamiltonian as the target may include contribution by the first Hamiltonian H1 to the third Hamiltonian H3 described above. The Hamiltonian as the target may also include terms which correspond to contribution of Zeeman energy, contribution of Dzyaloshinskii-Moriya interaction and the like.

The information related to the crystal structure of the material may include arbitrary information including: lattice-related information, for example, a lattice constant, a composition, a number of lattices, symmetry of the lattice (space group) and the like; and atom-related information such as a number of atoms included in the lattice, positions, valences, orbit states and states of electron spins of the atoms, symmetry (point group) around the atom and the like. This information may be recorded in any crystal structure database, may be described in a paper or the like, or may be obtained by various measurements such as X-ray diffraction experiments and the like. The model of the material includes the at least one site in which the atom is located.

The physical property simulation of the present embodiment includes first-principles calculation and finite temperature calculation. The physical property simulation may further include micromagnetic simulation, phase-field simulation, device simulation and the like.

### <First-Principles Calculation>

The first-principles calculation outputs first estimated data at the absolute zero point based on the acquired model of the material. The first-principles calculation in the present embodiment is executed by adopting density functional theory (DFT). Incidentally, the calculation method of the first estimated data at the absolute zero point is not limited to the first-principles calculation, but arbitrary methods such as a Hartree-Fock method, mean field approximation, a classical Monte Carlo method, a quantum Monte Carlo method and a variational Monte Carlo method can be adopted. The first-principles calculation of the present embodiment outputs: the saturation magnetization M0 at the absolute zero point; the coupling coefficient (the magnetic exchange coefficient Jij); and the magnetic anisotropy energy K at the absolute zero point, as the first estimated data at the absolute zero point. Incidentally, the magnetic anisotropy energy K may include: energy resulted from the uniaxial anisotropy; and energy resulted from the symmetry of the crystal structure.

### <Finite Temperature Calculation>

The finite temperature calculation outputs first estimated data at a finite temperature based on the output first estimated data at the absolute zero point. The first estimated data at the finite temperature includes: temperature dependence of the spontaneous magnetization M at the finite temperature; and temperature dependence K1 of the magnetic anisotropy energy at the finite temperature. The temperature dependence of the spontaneous magnetization M at the finite temperature includes a Curie temperature at which the spontaneous magnetization M becomes zero. A specific aspect of the finite temperature calculation is arbitrary, for example, the quantum Monte Carlo method, a first-principles molecular dynamics method, a first-principles lattice dynamics method or the like. In the present embodiment, the classical Monte Carlo method dares to be used as the finite temperature calculation. Thereby, a computational load for obtaining the first estimated data can be reduced, whereby reduction of a computation time and simulation of a larger system can be realized. Hereinafter, for convenience of explanation, the first estimated data at the absolute zero point and the first estimated data at the finite temperature may collectively be denoted just by the first estimated data. In other words, the first estimated data includes the first estimated data at the absolute zero point and the first estimated data at the finite temperature.

Incidentally, the above-described physical property simulation does not necessarily have to be executed by the information processing apparatus 2 itself, and may also be executed by an external device, for example, a supercomputer, cloud computing or the like. In this case, the information processing apparatus 2 may execute the said calculation indirectly by communicating with an external device.

### [Step S2]

Next, the processing proceeds to Step S2, in which the acquisition unit 231 acquires: the first estimated data calculated by the above-described physical property simulation; and the measured data obtained by the measurement on the material that is the target of the above-described physical property simulation.

### <Measured Data>

The measured data includes physical property values obtained by the measurement in a ferroic order state. The measured data includes at least a part of the temperature dependence of the spontaneous magnetization M. The temperature dependence of the spontaneous magnetization M includes, for example, the Curie temperature Tc as the phase transition temperature, the saturation magnetization M0 at the absolute zero point, and the like. Hereinafter, for convenience of explanation, the temperature dependence of the spontaneous magnetization M included in the measured data is denoted by temperature dependence ME of measured magnetization, the Curie temperature included in the measured data is denoted by a measured Curie temperature TcE, and the saturation magnetization M0 at the absolute zero point included in the measured data is denoted by measured saturation magnetization MOE.

The temperature dependence ME of the measured magnetization may include a value of the spontaneous magnetization M of the material at a finite temperature other than the phase transition temperature. Specifically, the temperature dependence of the spontaneous magnetization M may include a value of the spontaneous magnetization M at a finite temperature between the absolute zero point and the Curie temperature Tc. Further, the measured data does not have to include the Curie temperature Tc itself or the saturation magnetization M0 itself, and may be obtained by fitting of measurement results of the spontaneous magnetization M at plural temperatures. Moreover, the measured saturation magnetization M0E may also be a value which is obtained from the spontaneous magnetization M measured in the vicinity of the absolute zero point, or a value which is obtained from the said spontaneous magnetization M by an extrapolation method or the like. Further, the measured Curie temperature TcE is not limited to the temperature at a timing when the spontaneous magnetization M becomes exactly zero, and may also be a temperature obtained from temperatures before and after providing the spontaneous magnetization M to be zero. Such temperature dependence of the spontaneous magnetization M can be measured, by using, for example, a superconducting quantum interference device (SQUID) magnetometer.

The measured data may include susceptibility, which denotes response of the order parameter to a field conjugate to the order parameter. The field conjugate to the order parameter in the present embodiment is a magnetic field. In addition, the said susceptibility denotes magnetic susceptibility, in particular, complex magnetic susceptibility μ. The complex magnetic susceptibility μ is obtained from a result of measurement of the spontaneous magnetization M when, for example, an alternating magnetic field is applied. Magnetic field dependence of such spontaneous magnetization M can be measured by using, for example, the SQUID magnetometer described above. Hereinafter, for convenience of explanation, the complex magnetic susceptibility μ included in the measured data is denoted by measured magnetic susceptibility μE.

Further, the measured data may also include magnetic anisotropy energy K. The magnetic anisotropy energy K represents a difference in free energy generated when the ferromagnetic material is magnetized along an axis of easy magnetization and an axis of hard magnetization, respectively. The magnetic anisotropy energy K can be obtained by a following relational expression from, for example, a history of the magnetization on the magnetic field.

$$K = {\int }_{0}^{{M}_{s}} ( {H}_{ext}^{axis 1} - {H}_{ext}^{a xis 2} ) dM \qquad [ Formula 6 ]$$

A sign of M_s represents saturation magnetization at a certain temperature. A sign of H_ext represents the magnetic field. A sign of axis 1 denotes the axis of easy magnetization, and axis 2 denotes the axis of hard magnetization. The magnetic anisotropy energy K can be calculated from, for example, the saturation magnetization M_s at each measured temperature and the magnetic field dependence of the magnetization. Hereinafter, for convenience of explanation, the temperature dependence of the magnetic anisotropy energy K included in the second estimated data is denoted by temperature dependence K2 of second estimated magnetic anisotropy energy, and temperature dependence of the magnetic anisotropy energy K included in the measured data is denoted by temperature dependence KE of measured magnetic anisotropy energy. Measured data of the magnetic anisotropy energy K can be obtained by, for example, measuring the magnetic field dependence of the spontaneous magnetization M and integrating a hysteresis curve obtained from the said dependence.

### <Damping Constant α>

The damping constant α represents a degree of microscopic damping of the order parameter at the site. The damping constant α of the present embodiment is a Gilbert damping constant, which is used in a Landau-Lifshitz-Gilbert equation (LLG equation) described below, and represents, for example, a degree of suppression of precessional motion of the magnetization by an effective magnetic field H_eff.

$$\frac{dm}{dt} = - \frac{γ}{( 1 + {α}^{2} )} [ m \times {H}_{eff} ] - \frac{γ}{( 1 + {α}^{2} )} α [ m \times [ m \times {H}_{eff} ] ] \qquad [ Formula 7 ]$$

A sign of m represents local magnetization. A sign of H_eff represents an effective magnetic field that acts on the magnetization m. The effective magnetic field H_eff includes, for example, contribution of exchange energy by the first Hamiltonian H1, contribution of anisotropy energy by the second Hamiltonian H2, contribution by a Zeeman effect, contribution by demagnetization term and the like. A sign of γ is a Gyromagnetic constant. The damping constant α can be measured by, for example, ferromagnetic resonance measurement.

### [Step S3]

Next, the processing proceeds to Step S3, in which the data assimilation unit 232 executes data assimilation processing based on the first estimated data and the measured data that are acquired. Thereby, the data assimilation unit 232 calculates the second estimated data by assimilating the first estimated data to the measured data. The second estimated data may include a physical property which is equivalent to that of the first estimated data. The second estimated data includes, for example, temperature dependence of the spontaneous magnetization M on which the data assimilation is executed, the Curie temperature Tc, the magnetic exchange coefficient Jij, the magnetic anisotropy energy K, the exchange stiffness constant A and the like. Detail of the data assimilation processing will be described below. Hereinafter, for convenience of explanation, the temperature dependence of the spontaneous magnetization M included in the second estimated data is denoted by temperature dependence M2 of second spontaneous magnetization, and the Curie temperature Tc included in the second estimated data is denoted by a second estimated Curie temperature Tc2. In addition, the magnetic exchange coefficient Jij included in the second estimated data is denoted by a second estimated magnetic exchange coefficient Jij2. Further, the temperature dependence of the magnetic anisotropy energy K included in the second estimated data is denoted by the temperature dependence K2 of the second estimated magnetic anisotropy energy.

### [Step S4]

Next, the processing proceeds to Step S4, in which the output unit 234 outputs the coupling coefficient, on which the first data assimilation processing is executed, as the second estimated data. The output second estimated data can be used for arbitrary purposes such as, for example, an input parameter for micromagnetic simulation and the like. Specifically, the output unit 234 executes the micromagnetic simulation by substituting the first estimated data and the second estimated data into the LLG equation.

### 3.2. Flow of Data Assimilation Processing

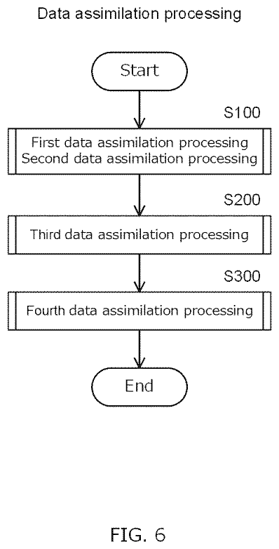

Next, the data assimilation processing in Step S3 will be described. FIG. 6 is a flowchart that represents a flow of the data assimilation processing.

### [Step S100]

Firstly, in Step S100, the data assimilation unit 232 executes processing including: first data assimilation processing for the coupling coefficient based on the first estimated data and the measured data that are acquired; and second data assimilation processing for the temperature dependence M1 of the spontaneous magnetization M included in the acquired first estimated data.

In the first data assimilation processing, the data assimilation unit 232 multiplies: a ratio of the measured reference value to the estimated reference value; with the coupling coefficient included in the acquired first estimated data according to a degree that represents dependence of the estimated reference value on the coupling coefficient. Thereby, the data assimilation unit 232 executes the data assimilation on the coupling coefficient.

### <Measured Reference Value>

The measured reference value is a physical property value to be used in the data assimilation processing (particularly in the first data assimilation processing). The measured reference value includes at least one of: the measured Curie temperature TcE as the phase transition temperature; and the measured saturation magnetization M0E as the saturation value.

### <Estimated Reference Value>

The estimated reference value is a value corresponding to the measured reference value among: the first estimated Curie temperature Tc1 as the phase transition temperature; and the first estimated saturation magnetization M01 as the saturation value. When the measured reference value includes the measured Curie temperature TcE, the estimated reference value includes the first estimated Curie temperature Tc1. Whereas, when the measured reference value includes the measured saturation magnetization M0E, the estimated reference value includes the first estimated saturation magnetization M01.

Further, when the measured data includes the measured reference value, the data assimilation unit 232 executes the second data assimilation processing by multiplying the temperature dependence M1 of the spontaneous magnetization M included in the acquired first estimated data, based on the ratio of the measured reference value to the estimated reference value.

As a result of the processing in Step S100, the output unit 234 outputs the second estimated magnetic exchange coefficient Jij2 and the temperature dependence M2 of the second spontaneous magnetization. In addition, as a result of the processing in Step S100, the output unit 234 of the present embodiment calculates temperature dependence of the exchange stiffness constant A based on the magnetic exchange coefficient Jij included in the estimated data and the temperature dependence of the saturation magnetization M0, and output the temperature dependence of the exchange stiffness constant A as the second estimated data.

### [Step S200]

Next, the processing proceeds to Step S200, in which the data assimilation unit 232 executes third data assimilation processing on the temperature dependence of the anisotropy energy included in the first estimated data (the temperature dependence K1 of the first estimated magnetic anisotropy energy in the present embodiment), based on at least one of: the coupling coefficient on which the first data assimilation processing is executed (the second estimated magnetic exchange coefficient Jij2 in the present embodiment); and the temperature dependence of the order parameter on which the second data assimilation processing is executed (the temperature dependence M2 of the second spontaneous magnetization in the present embodiment). Thereby, the output unit 234 outputs the temperature dependence of the anisotropy energy on which the third data assimilation processing is executed (the temperature dependence K2 of the second estimated magnetic anisotropy energy in the present embodiment) as the second estimated data.

### [Step S300]

Next, the processing proceeds to Step S300, in which the processor 23 executes physical property simulation based on the second estimated data, which is calculated in Steps S100 and S200. Thereby, the output unit 234 outputs temperature dependence of a first damping constant α1. Then, the data assimilation unit 232 executes fourth data assimilation processing on the output first damping constant α1. Thereby, a second damping constant α2 on which the data assimilation is executed is obtained as the second estimated data.

Using the second estimated data output due to these processing, micromagnetic simulation and the like are executed.

### 3.3. Detail of Processing in Step S100

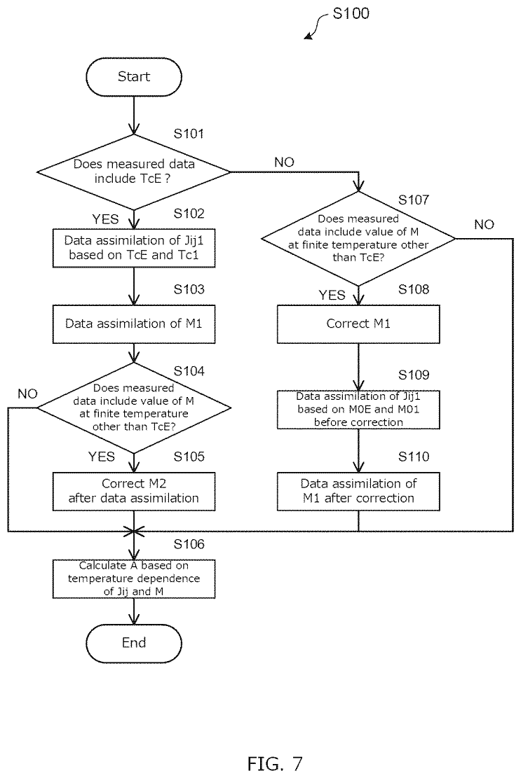

Next, details of the processing in Step S100 described above will be explained. FIG. 7 is a flowchart that represents the details of the processing in Step S100.

### [Step S101]

Firstly, in Step S101, the processor 23 judges whether the acquired measured data includes the measured Curie temperature TcE or not. The said judgment may be performed depending on an input by the user, or performed in accordance with a format of the measured data.

### [Step S102]

When the measured data includes the measured Curie temperature TCE (when the judgment result of Step S101 is positive), the processing proceeds to Step S102, in which the data assimilation unit 232 executes data assimilation on the first estimated magnetic exchange coefficient Jij1, based on the measured Curie temperature TcE as the measured reference value and the first estimated Curie temperature Tc1 as the estimated reference value corresponding to the said measured Curie temperature TcE. The processing in Step S102 for executing the data assimilation on the first estimated magnetic exchange coefficient Jij1 can be considered as the first data assimilation processing in the case where the estimated reference value is the first estimated Curie temperature Tc1. Also, the processing in Step S102 can be considered as the first data assimilation processing.

In detail, the data assimilation unit 232 calculates a ratio TcE/Tc1 of the measured Curie temperature TcE to the first estimated Curie temperature Tc1. Next, the data assimilation unit 232 calculates the second estimated magnetic exchange coefficient Jij2 by multiplying an exponentiation of the said ratio TcE/Tc1 with the first estimated magnetic exchange coefficient Jij1, based on the dependence of the magnetic exchange coefficient Jij on the Curie temperature Tc. The dependence of the magnetic exchange coefficient Jij on the Curie temperature Tc includes, for example, a proportional degree of the Curie temperature Tc with respect to the magnetic exchange coefficient Jij. In the present embodiment, if considering only nearest neighbor interaction, the magnetic exchange coefficient Jij is proportional to a first order of the Curie temperature Tc, whereby the data assimilation unit 232 calculates the second estimated magnetic exchange coefficient Jij2 by multiplying a first power of the ratio TcE/Tc1 with the first estimated magnetic exchange coefficient Jij1. Thereby, the data assimilation unit 232 executes the first data assimilation so as to able to replace contribution of the first estimated Curie temperature Tc1 included in the first estimated magnetic exchange coefficient Jij1 substantially with contribution of the measured Curie temperature TcE, thus obtaining the second estimated magnetic exchange coefficient Jij2, which provides less discrepancy with an experimental fact than that of the first estimated magnetic exchange coefficient Jij1.

### [Step S103]

Next, the processing proceeds to Step S103, in which data assimilation is executed on the temperature dependence M1 of the first spontaneous magnetization based on the ratio TcE/Tc1 of the measured Curie temperature TcE to the first estimated Curie temperature Tc1. Thereby, the temperature dependence M2 of the second spontaneous magnetization having the Curie temperature Tc, which is more in accordance with an experimental fact than that of the temperature dependence M1 of the first spontaneous magnetization, can be obtained. In this case, when a difference between: the second estimated Curie temperature Tc2 obtained again by using the second estimated magnetic exchange coefficient Jij2; and the measured Curie temperature TcE is larger than a certain threshold value, the processing may repeat Step S102 again in which the second estimated Curie temperature Tc2 is adopted as the first estimated Curie temperature Tc1. Also, Step S103 of executing the data assimilation on the temperature dependence M1 of the first spontaneous magnetization can be considered as one of the second data assimilation processing in the present embodiment.

In the present embodiment, the data assimilation unit 232 executes finite temperature calculation (for example, the classical Monte Carlo calculation) again using the coupling coefficient (the second estimated magnetic exchange coefficient Jij2) on which the first data assimilation processing is executed. Thereby, when executing the finite temperature calculation adopting the second estimated magnetic exchange coefficient Jij2, the processing can be simplified compared with that of adopting a method different from the finite temperature calculation, which is used for obtaining the first estimated magnetic exchange coefficient Jij1. Incidentally, the method of the finite temperature calculation adopted in Step S103 may be different from the method of the finite temperature calculation adopted in Step S1. Since the second estimated magnetic exchange coefficient Jij2 is obtained based on the ratio TcE/Tc1, the processing based on the second estimated magnetic exchange coefficient Jij2 can be considered as the processing based on the said ratio TcE/Tc1.

When executing the finite temperature calculation again, at least a part of the data calculated in the previous finite temperature calculations (the first estimated data or the like) may be used as a constraint condition. Thereby, a calculation range can be limited, thereby suppressing divergence of a calculation amount.

The specific aspect of the data assimilation on the temperature dependence M1 of the first spontaneous magnetization is not limited to the above. For example, the data assimilation unit 232 may execute the data assimilation on the temperature dependence M1 of the first spontaneous magnetization by converting a temperature as a variable included in the temperature dependence M1 of the first spontaneous magnetization, based on the said ratio TcE/Tc1. In detail, the data assimilation unit 232 executes conversion of a temperature axis with respect to the temperature dependence M1 of the first spontaneous magnetization. The specific aspect of such correction is arbitrary, and, for example, the conversion of the temperature axis is executed based on a following relational expression. Incidentally, T denotes the temperature as the variable.

$$M 2 ( T \times \frac{TcE}{Tc 1} ) = M 1 ( T ) \qquad [ Formula 8 ]$$

The said conversion corresponds to a change of a scale of the temperature axis. Therefore, the temperature dependence M2 of the second spontaneous magnetization, of which the first estimated Curie temperature Tc1 is adjusted to the measured Curie temperature TcE, while maintaining qualitative properties of the temperature dependence M1 of the first spontaneous magnetization, can be obtained.

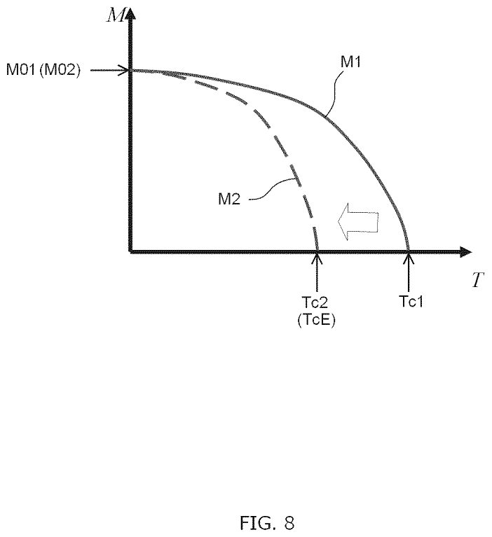

FIG. 8 is a view that illustrates a change in the temperature dependence of the spontaneous magnetization M due to the data assimilation in Step S103. The first estimated Curie temperature Tc1, which is obtained by the physical property simulation in Step S100, is evaluated to be higher than the measured Curie temperature TcE. As a result of the above-described processing in Step S103, the temperature dependence M1 of the first spontaneous magnetization is reduced along the temperature axis. Thereby, the temperature dependence M2 of the second spontaneous magnetization, of which the second estimated Curie temperature Tc2 can match the measured Curie temperature TcE, while maintaining the qualitative properties of the temperature dependence M1 of the first spontaneous magnetization, can be obtained. Incidentally, in the processing in Step S103, the second estimated saturation magnetization M02 is subjected to the data assimilation to match the first estimated saturation magnetization M01. Thereby, it can be suppressed that, when the measured data is present only in the vicinity of the measured Curie temperature TcE, the data assimilation using the said measured data affects the first estimated data in a region where the experimental fact is not verified by the measurement.

### [Step S104]

As shown in FIG. 7, the process then proceeds to Step S104, in which the processor 23 judges whether the measured data includes at least one value of the order parameter in the material at a finite temperature other than the phase transition temperature or not. In the present embodiment, the correction unit 233 judges whether the measured data includes at least one value of the spontaneous magnetization M at a finite temperature other than the measured Curie temperature TcE or not. In other words, the correction unit 233 judges whether the temperature dependence ME of the measured magnetization includes a value other than the measured reference values (that is, a value other than the measured Curie temperature Tc or the measured saturation magnetization MOE) or not.

### [Step S105]

When the measured data includes at least one value of the order parameter in the material at a finite temperature other than the phase transition temperature (when the judgment result of Step S104 is positive), the processing proceeds to Step S105, and the correction unit 233 further corrects the temperature dependence of the order parameter included in the estimated data, based on the said value of the order parameter of the material at the finite temperature. In the present embodiment, the correction unit 233 corrects the temperature dependence M2 of the second spontaneous magnetization obtained by the processing in Step S103, based on the value of the spontaneous magnetization M at a finite temperature included in the temperature dependence ME of the measured magnetization. The specific aspect of the said correction is arbitrary, and, for example, the correction unit 233 corrects the temperature dependence M2 of the second spontaneous magnetization, by fitting the temperature dependence M2 of the second spontaneous magnetization based on the said value of the spontaneous magnetization M at the finite temperature by a least squares method, a maximum likelihood method or the like. At this time, the second estimated Curie temperature Tc2 may be fixed as a constraint condition of the said correction. Thereby, the temperature dependence M2 of the second spontaneous magnetization, which is more in accordance with the experimental fact, can be obtained while maintaining the result of the data assimilation on the Curie temperature Tc, by the processing in Step S103. The correction unit 233 updates the corrected temperature dependence M2 of the second spontaneous magnetization as the latest temperature dependence M2 of the second spontaneous magnetization. The acquisition unit 231 can also obtain the second estimated saturation magnetization M02 from the value of the temperature dependence M2 of the second spontaneous magnetization at the absolute zero point.

### [Step S106]

Next, the processing proceeds to Step S106, in which the data assimilation unit 232 calculates the temperature dependence of the exchange stiffness constant A based on the magnetic exchange coefficient Jij and the temperature dependence of the spontaneous magnetization M which are included in the estimated data. The data assimilation unit 232 may calculate the temperature dependence of the exchange stiffness constant A using the above-mentioned relational expression. If the processing in Step S105 has been executed, the data assimilation unit 232 calculates the temperature dependence of the exchange stiffness constant A, based on the second estimated magnetic exchange coefficient Jij2 obtained in Step S102 and the temperature dependence M2 of the second spontaneous magnetization corrected in Step S105. Then, the output unit 234 outputs the temperature dependence of the exchange stiffness constant A. The output unit 234 outputs latest data of the various calculated parameters as the second estimated data. If the processing proceeds via Step S105, the second estimated data includes: the second estimated magnetic exchange coefficient Jij2 obtained in Step S102; the temperature dependence M2 of the second spontaneous magnetization after the correction obtained in Step S105; and the temperature dependence of the exchange stiffness constant A obtained in Step S106. When the processing in Step S106 is completed, the processor 23 completes the processing of Step S100.

Whereas, when the measured data does not include a value of the order parameter in the material at the finite temperature other than the phase transition temperature (when the judgment result of Step S104 is negative), Step S105 is omitted and the processing proceeds to Step S106. In this case, the second estimated data include: the second estimated magnetic exchange coefficient Jij2 obtained in Step S102; the temperature dependence M2 of the second spontaneous magnetization obtained in Step S103; and the temperature dependence of the exchange stiffness constant A obtained in Step S106.

### [Step S107]

Whereas, when the measured data does not include the measured Curie temperature TcE (when the judgment result of Step S101 is negative), the processing proceeds to Step S107, in which the processor 23 judges whether the measured data includes at least one value of the order parameters (the spontaneous magnetization M) of the material at a finite temperature other than the phase transition temperature (the Curie temperature Tc) or not. Details of the judgment processing are similar to those in Step S104.

### [Step S108]

When the measured data includes at least one value of the order parameters of the material at the finite temperature other than the phase transition temperature (including at the absolute zero point and a vicinity thereof) (when the judgment result of Step S107 is positive), the processing proceeds to Step S108, in which the correction unit 233 further corrects the temperature dependence of the order parameter included in the estimated data, based on the said value of the order parameter in the material at the finite temperature. In the present embodiment, the correction unit 233 corrects the temperature dependence M1 of the first spontaneous magnetization acquired by the processing in Step S1, based on the value of the spontaneous magnetization M at the finite temperature included in the temperature dependence ME of the measured magnetization. Thereby, the acquisition unit 231 acquires at least one of the measured Curie temperature TcE and the measured saturation magnetization M0E (that is, the measured reference value) from the corrected temperature dependence M1 of the first spontaneous magnetization. The specific aspect of the said correction is arbitrary, and, for example, the correction unit 233 corrects the temperature dependence M2 of the second spontaneous magnetization, by fitting the temperature dependence M2 of the second spontaneous magnetization by the least squares method, the maximum likelihood method or the like. In the correction in Step S108, the second estimated saturation magnetization M02 is not fixed. Thereby, the temperature dependence M2 of the second spontaneous magnetization, which is more in accordance with the experimental fact, can be obtained, whereby more accurate estimated data of the saturation magnetization M0 can be obtained. If the measured saturation magnetization M0E has not been obtained experimentally, the correction unit 233 uses this estimated saturation magnetization M0 substantially as the measured saturation magnetization MOE. If the measured saturation magnetization M0E has been experimentally obtained, the correction unit 233 uses the measured saturation magnetization M0E directly as it is. The correction unit 233 updates the corrected temperature dependence M1 of the first spontaneous magnetization as the latest temperature dependence M2 of the second spontaneous magnetization. The corrected temperature dependence M1 of the first spontaneous magnetization includes the first estimated saturation magnetization M01 and the first estimated Curie temperature Tc1.

### [Step S109]

Next, data assimilation is executed on the first estimated magnetic exchange coefficient Jij1 based on: the measured saturation magnetization M0E (in other words, the first estimated saturation magnetization M01 after the correction) as the measured reference value; and the first estimated saturation magnetization M01 before the correction as the estimated reference value. The processing in Step S109 for executing the data assimilation on the first estimated magnetic exchange coefficient Jij1 can be considered also as the first data assimilation processing in the case where the estimated reference value is the second estimated saturation magnetization M02.

In detail, the data assimilation unit 232 calculates the ratio M0E/M01 of the measured saturation magnetization M0E to the first estimated saturation magnetization M01 before the correction. Next, the data assimilation unit 232 calculates the second estimated magnetic exchange coefficient Jij2 by multiplying an exponentiation of the said ratio M0E/M01 with the first estimated magnetic exchange coefficient Jij1, based on dependence of the magnetic exchange coefficient Jij on the spontaneous magnetization M. The dependence of the magnetic exchange coefficient Jij on the spontaneous magnetization M includes, for example, a proportional degree of the spontaneous magnetization M with respect to the magnetic exchange coefficient Jij1. In the present embodiment, since the magnetic exchange coefficient Jij is proportional to a second order of the spontaneous magnetization M, the data assimilation unit 232 calculates the second estimated magnetic exchange coefficient Jij2 by multiplying a square of the ratio M0E/M01 with the first estimated magnetic exchange coefficient Jij1. Thereby, the data assimilation unit 232 executes the first data assimilation so as to replace contribution of the temperature dependence M1 of the first spontaneous magnetization included in the first estimated magnetic exchange coefficient Jij1 substantially with contribution of the measured saturation magnetization M0E, thus obtaining the second estimated magnetic exchange coefficient Jij2, which provides less discrepancy with the experimental fact than that of the first estimated magnetic exchange coefficient Jij1.

### [Step S110]

Next, the processing proceeds to Step S110, in which the data assimilation unit 232 executes data assimilation on the temperature dependence M1 of the first spontaneous magnetization after the correction, based on the ratio M0E/M01 of the measured saturation magnetization M0E to the first estimated saturation magnetization M01 after the correction. Thereby, the temperature dependence M2 of the second spontaneous magnetization having the saturation magnetization M0, which is more in accordance with the experimental fact than that of the temperature dependence M1 of the first spontaneous magnetization, can be obtained. Step S110 of executing the data assimilation on the temperature dependence M1 of the first spontaneous magnetization can also be considered as one of the second data assimilation processing of the present embodiment.

In the present embodiment, the data assimilation unit 232 executes finite temperature calculation (for example, the classical Monte Carlo calculation) again using the coupling coefficient (the second estimated magnetic exchange coefficient Jij2) on which the first data assimilation processing is executed, similarly to Step S103. Thereby, when executing the finite temperature calculation using the second estimated magnetic exchange coefficient Jij2, the processing can be simplified more than that of calculation in a method different from the finite temperature calculation, which is used for obtaining the first estimated magnetic exchange coefficient Jij1.

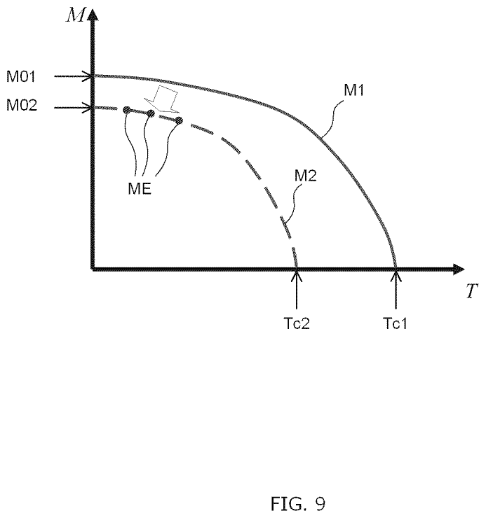

FIG. 9 is a view that illustrates a change in the temperature dependence of the spontaneous magnetization M due to the data assimilation in Step S110. The temperature dependence M1 of the first spontaneous magnetization which is obtained by the physical property simulation in Step S100 is evaluated to be higher than the temperature dependence ME of the measured magnetization. As a result of the processing in Step S110 described above, a deviation between the temperature dependence M1 of the first spontaneous magnetization and the temperature dependence ME of the measured magnetization can be suppressed, while maintaining the qualitative properties of the temperature dependence M1 of the first spontaneous magnetization. Incidentally, in the processing in Step S110, unlike in the processing in Step S103, the second estimated saturation magnetization M02 and the first estimated saturation magnetization M01 may be different.

Incidentally, the specific aspect of the data assimilation on the temperature dependence M1 of the first spontaneous magnetization is not limited to the above. For example, the data assimilation unit 232 may also execute the data assimilation on the temperature dependence M1 of the first spontaneous magnetization by converting the temperature as a variable included in the temperature dependence M1 of the first spontaneous magnetization, based on the said ratio M0E/M01. In detail, the data assimilation unit 232 executes the conversion of the temperature axis with respect to the temperature dependence M1 of the first spontaneous magnetization. The specific aspect of such correction is arbitrary, and, for example, the conversion of the temperature axis is executed based on a following relational expression. Incidentally, T denotes the temperature as the variable.

$$M 2 ( T \times {( \frac{M 0 E}{M 01} )}^{2} ) = M 1 ( T ) \qquad [ Formula 9 ]$$

The said conversion corresponds to a change of the scale of the temperature axis. Therefore, the temperature dependence M2 of the second spontaneous magnetization, of which the first estimated saturation magnetization M01 is adjusted to the measured saturation magnetization M0E, while maintaining the qualitative properties of the temperature dependence M1 of the first spontaneous magnetization, can be obtained.

### [Step S106]

As shown in FIG. 7, the processing then proceeds to Step S106, in which the data assimilation unit 232 calculates the temperature dependence of the exchange stiffness constant A based on the magnetic exchange coefficient Jij included in the estimated data and the temperature dependence of the spontaneous magnetization M. If the processing has proceeded via Steps S108 to S110, the data assimilation unit 232 may calculate the temperature dependence of the exchange stiffness constant A based on the second estimated magnetic exchange coefficient Jij2 obtained in Step S109 and the temperature dependence M2 of the second spontaneous magnetization obtained in Step S110.

Incidentally, when the judgment result of Step S107 is negative (that is, when the measured data includes neither the measured Curie temperature TcE or the value of the spontaneous magnetization M at the finite temperature other than the measured Curie temperature TcE), Steps S108 to S110 are omitted, and the processing proceeds to Step S106. In this case, the first data assimilation processing and the second data assimilation processing are omitted, and the processor 23 calculates the exchange stiffness constant A, based on the first estimated magnetic exchange coefficient Jij1 and the temperature dependence M1 of the first spontaneous magnetization which are acquired in Step S2.

### 3.3. Details of Processing in Step S200

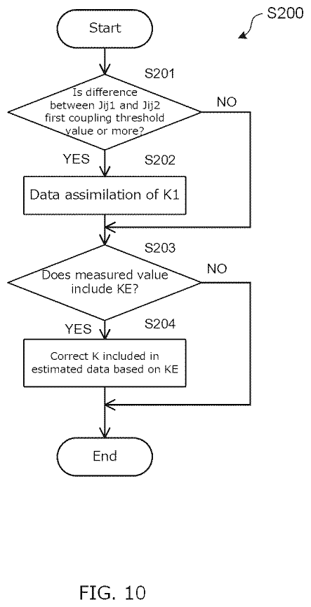

Next, details of the processing in Step S200 described above will be explained. FIG. 10 is a flowchart that represents the details of the processing in Step S200.

### [Step S201]

Firstly, in Step S201, the processor 23 judges whether a difference between: the coupling coefficient included in the first estimated data (the first estimated magnetic exchange coefficient Jij1); and the coupling coefficient on which the data assimilation processing (in detail, the first data assimilation processing) is executed (the second estimated magnetic exchange coefficient Jij2) is a first coupling threshold value or more, or not. Incidentally, a format of the difference between the above-described coupling coefficients is arbitrary, and may be subtraction, an amount of change, a rate of change, a ratio or the like. The first coupling threshold value can be set arbitrarily according to accuracy required for the second estimated data.

### [Step S202]

When the difference between the first estimated magnetic exchange coefficient Jij1 and the second estimated magnetic exchange coefficient Jij2 is the first coupling threshold value or more (that is, when the judgment result of Step S201 is positive), the processing proceeds to Step S202, in which the data assimilation unit 232 executes data assimilation on the temperature dependence K1 of the first estimated magnetic anisotropy energy, based on at least one value of the second estimated data calculated in Step S100. The processing in Step S202 for executing the data assimilation on the temperature dependence K1 of the first estimated magnetic anisotropy energy can be considered as one of the third data assimilation processing of the present embodiment.

In the present embodiment, finite temperature calculation is executed again using: the coupling coefficient on which the first data assimilation processing is executed (the second estimated magnetic exchange coefficient Jij2); and the temperature dependence of the order parameter on which the second data assimilation processing is executed (the temperature dependence M2 of the second spontaneous magnetization). Thereby, the temperature dependence K2 of the second estimated magnetic anisotropy energy, on which the information on the magnetic exchange coefficient Jij that is more in accordance with an experimental fact than that of the temperature dependence K1 of the first estimated magnetic anisotropy energy is reflected, can be obtained. In the present embodiment, the method of the finite temperature calculation used in Step S202 is similar to that in Step S1, and may also be different.

Incidentally, the specific aspect of the data assimilation on the temperature dependence K1 of the first estimated magnetic anisotropy energy is not limited to the above. For example, the data assimilation unit 232 may execute the data assimilation on the temperature dependence K1 of the first estimated magnetic anisotropy energy by converting the temperature as the variable included in the temperature dependence K1 of the first estimated magnetic anisotropy energy, based on the ratio Tc2/Tc1 of the second estimated Curie temperature Tc2 to the first estimated Curie temperature Tc1. In detail, the data assimilation unit 232 converts the temperature axis of the temperature dependence K1 of the first estimated magnetic anisotropy energy. The specific aspect of the said correction is arbitrary, and the conversion of the temperature axis is executed based on, for example, a following relational expression.

$$K 2 ( T \times \frac{Tc 2}{Tc 1} ) = K 1 ( T ) \qquad [ Formula 10 ]$$

The said conversion corresponds to a change in the scale of the temperature axis. Therefore, the temperature dependence K2 of the second estimated magnetic anisotropy energy, of which the Curie temperature Tc is adjusted to the second estimated Curie temperature Tc2 on which the experimental fact is reflected more than that of the first estimated Curie temperature Tc1, while maintaining the qualitative properties of the temperature dependence K1 of the first estimated magnetic anisotropy energy, can be obtained.

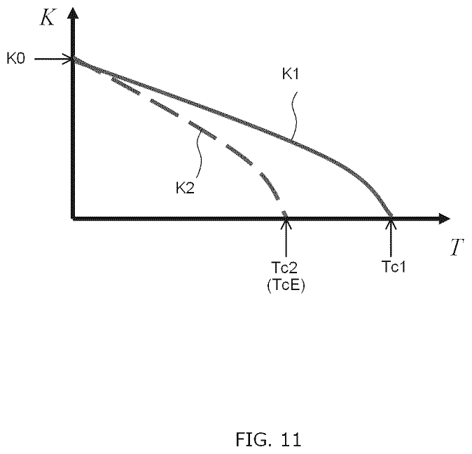

FIG. 11 is a view that illustrates a change in the temperature dependence of the magnetic anisotropy energy K due to the data assimilation in Step S202. The temperature dependence K1 of the first estimated magnetic anisotropy energy which is obtained by the physical property simulation in Step S100 is evaluated to be higher than the temperature dependence KE of the measured magnetic anisotropy energy. As a result of the above-described processing in Step S103, the temperature dependence K1 of the first estimated magnetic anisotropy energy is reduced along the temperature axis. Thereby, the temperature dependence K2 of the second estimated magnetic anisotropy energy, of which the second estimated Curie temperature Tc2 can match the measured Curie temperature TcE, while maintaining the qualitative properties of the temperature dependence K1 of the first estimated magnetic anisotropy energy, can be obtained. Incidentally, in the processing in Step S202, the magnetic anisotropy energy K02 at the absolute zero point included in the second estimated data is subjected to the data assimilation to match the magnetic anisotropy energy K01 at the absolute zero point included in the first estimated data.

As shown in FIG. 10, the processing subsequently proceeds from Step S202 to Step S203. Incidentally, when the difference between: the coupling coefficient included in the first estimated data; and the coupling coefficient, on which the first data assimilation processing is executed, is less than the first coupling threshold value (that is, when the judgment result of Step S201 is negative), the processing in Step S202 is omitted and proceeds to Step S203.

### [Step S203]

Next, in Step S203, the processor 23 judges whether the measured data includes the temperature dependence of the anisotropy energy (the temperature dependence KE of the measured magnetic anisotropy energy) or not.

### [Step S204]

When the measured data includes the temperature dependence of the anisotropy energy (the temperature dependence KE of the measured magnetic anisotropy energy) (when the judgment result of Step S203 is positive), the correction unit 233 corrects the temperature dependence of the anisotropy energy included in the estimated data, based on the measurement result of the said temperature dependence of the anisotropy energy (the temperature dependence KE of the measured magnetic anisotropy energy). If the processing in Step S202 has been executed, a target to be corrected in Step S204 is the temperature dependence K2 of the second estimated magnetic anisotropy energy. On the other hand, if the processing in Step S202 has been omitted, the target to be corrected in Step S204 is the temperature dependence K1 of the first estimated magnetic anisotropy energy.

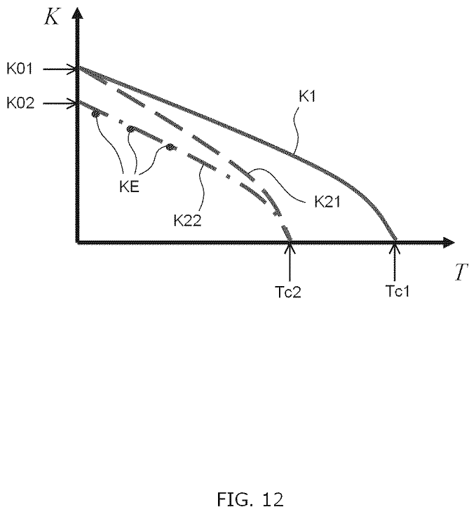

Herein, the correction of the temperature dependence K2 of the second estimated magnetic anisotropy energy will be described in more detail. FIG. 12 is a view that illustrates a change in the temperature dependence K2 of the second magnetic anisotropy energy K due to the correction in Step S204. In FIG. 12, the temperature dependence K2 of the second estimated magnetic anisotropy energy before the correction in Step S203 is represented as K21, and the temperature dependence K2 of the second estimated magnetic anisotropy energy after the correction in Step S203 is represented as K22.

The correction unit 233 corrects the temperature dependence K1 of the first estimated magnetic anisotropy energy or the temperature dependence K2 of the second estimated magnetic anisotropy energy, based on the value of the magnetic anisotropy energy K at a finite temperature included in the temperature dependence KE of the measured magnetic anisotropy energy. The said correction is executed by, for example, the least squares method, the maximum likelihood method or the like. The temperature dependence K21 of the second estimated magnetic anisotropy energy before the correction has already been subjected to the data assimilation on the temperature axis in Step S202. Therefore, in the correction in Step S204, the correction unit 233 fixes the second estimated Curie temperature Tc2 while correcting the temperature dependence K2 of the second estimated magnetic anisotropy energy. Thereby, consistency with the experimental fact can be maintained. Whereas, in the correction in Step S204, the correction unit 233 does not fix the magnetic anisotropy energy K0 at the absolute zero point to the magnetic anisotropy energy K01 at the absolute zero point included in the first estimated data. Thereby, the magnetic anisotropy energy K0 at the absolute zero point, which is more in accordance with the experimental fact, is likely to be obtained. In FIG. 12, the temperature dependence K21 of the second estimated magnetic anisotropy energy before the correction and the temperature dependence K22 of the second estimated magnetic anisotropy energy after the correction have substantially the same second estimated Curie temperature Tc2. Whereas, as a result of the correction based on the temperature dependence KE of the measured magnetic anisotropy energy, the magnetic anisotropy energy K02 at the absolute zero point after the correction becomes lower than the magnetic anisotropy energy K01 at the absolute zero point before the correction.

Incidentally, when the measured data does not include the measured Curie temperature TcE, the magnetic anisotropy energy K01 at the absolute zero point included in the first estimated data is preferably fixed while correcting the temperature dependence K2 of the second estimated magnetic anisotropy energy. Thereby, divergence of the calculation amount can be suppressed.

As shown in FIG. 10, after completion of the processing in Step S204, the processing in Step S200 is completed. On the other hand, when the measured data does not include the measurement result of the temperature dependence of the anisotropy energy (the temperature dependence KE of the measured magnetic anisotropy energy) (when the judgment result in Step S203 is negative), the processing in Step S204 is omitted, whereby the processing in Step S200 is completed.

### 3.4. Details of Processing in Step S300

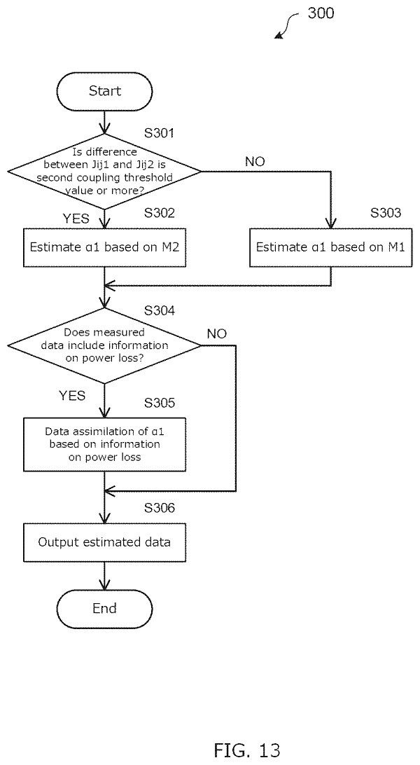

Next, details of the processing in Step S300 described above will be explained. FIG. 13 is a flowchart that represents details of processing in Step S300.

### [Step S301]

Firstly, in Step S301, the processor 23 judges whether a difference between the first estimated magnetic exchange coefficient Jij1 and the second estimated magnetic exchange coefficient Jij2 is a second coupling threshold value or more, or not. The second coupling threshold value can be set appropriately according to required accuracy and calculation resources. Incidentally, the difference between the first estimated magnetic exchange coefficient Jij1 and the second estimated magnetic exchange coefficient Jij2 has a correlation with a difference between the temperature dependence M1 of the first spontaneous magnetization and the temperature dependence M2 of the second spontaneous magnetization. Therefore, the judgment in Step S301 is synonymous with judgment based on the difference between the temperature dependence M1 of the first spontaneous magnetization and the temperature dependence M2 of the second spontaneous magnetization.

### [Step S302]

When the difference between the coupling coefficient included in the first estimated data (the first estimated magnetic exchange coefficient Jij1) and the coupling coefficient included in the second estimated data (the second estimated magnetic exchange coefficient Jij2) is the second coupling threshold value or more (when the judgment result of Step S301 is positive), the processing proceeds to Step S302, in which the processor 23 executes the physical property simulation based on the second estimated data. Specifically, as the physical property simulation, the processor 23 calculates a damping constant α0 at the absolute zero point by the first-principles calculation, and executes finite temperature calculation by inputting the second estimated data such as the second estimated magnetic exchange coefficient Jij2 and the second estimated saturation magnetization M02 in addition to the said damping constant α0. As a result, the output unit 234 outputs the first damping constant α1. Thereby, the damping constant α, which is more in accordance with an experimental fact than that in the case of calculating α1 or the like using the first estimated data, can be obtained. Incidentally, the specific method of the first-principles calculation in Step S302 is preferably algorithm based on a linear response theory included in, for example, a SPR-KKR program. Incidentally, the specific method of the said calculation is not limited to this, and may employ algorithm in an Akai-KKR program. Thereafter, the processing proceeds to Step S304.

Incidentally, the difference between the first estimated magnetic exchange coefficient Jij1 and the second estimated magnetic exchange coefficient Jij2 indicates that the experimental fact is different from an ideal conditions underlying the simulation in Step S1, by a tolerable amount or more.

### [Step S303]

Whereas, when the difference between the coupling coefficient included in the first estimated data (the first estimated magnetic exchange coefficient Jij1) and the coupling coefficient on which the first data assimilation processing is executed (the second estimated magnetic exchange coefficient Jij2) is less than the second coupling threshold value, the processor 23 executes the physical property simulation based on the first estimated data. Specifically, the processor 23 executes the finite temperature calculation by inputting the first estimated data such as the first estimated magnetic exchange coefficient Jij1 and the first estimated saturation magnetization M01, as the physical property simulation. As a result, the output unit 234 outputs the first damping constant α1. Thereafter, the processing proceeds to Step S304.

As described above, by omitting the calculation of the damping constant α in the physical property simulation in Step S1 and changing the calculation aspect of the damping constant α according to the change of the magnetic exchange coefficient Jij due to the data assimilation, calculation resources can be saved from overlapped calculation.

Incidentally, if the first damping constant α1 and the like are calculated by executing the physical property simulation based on the first estimated data in Step S1, the processing in Step S303 may be omitted.

### [Step S304]

In Step S304, the processor 23 judges whether the measured data includes information related to power loss P in the material (ferromagnetic material), which is caused by application of a field conjugate to the order parameter, or not. In the present embodiment, the processor 23 judges whether the measured data includes measured magnetic susceptibility μE or not. The power loss P includes, for example, eddy current loss P_E and hysteresis loss P_H. The eddy current loss P_E is represented as follows.

$${P}_{E} = \frac{{π}^{2} {Vd}^{2} {f}^{2} {H}^{2}}{6 ρ ( T )} \qquad [ Formula 11 ]$$

A sign of V denotes a volume of the material, d denotes a thickness of the material, and f denotes a frequency of a magnetic field H. The data assimilation unit 232 can calculate resistivity ρ by executing the physical property simulation based on the latest estimated data (the temperature dependence M2 of the second spontaneous magnetization, the second estimated magnetic exchange coefficient Jij2, the temperature dependence K2 of the second estimated magnetic anisotropy energy, and the like). Thus, the power loss P can be calculated by executing the physical property simulation based on the latest estimated data.

Further, the hysteresis loss P_H can be represented as follows.

$${P}_{H} = π {VfH}^{2} μ 2 ( T , H , f ) \qquad [ Formula 12 ]$$

A sign of μ2 is an imaginary number component of complex magnetic susceptibility μ. The data assimilation unit can calculate the imaginary number component μ2 of the complex magnetic susceptibility μ from the hysteresis loss P_H based on the above-described relation. Therefore, the complex magnetic susceptibility μ (in particular, the imaginary number component μ2) can be included in the information related to the power loss P. Hereinafter, for convenience of explanation, the hysteresis loss P_H included in the measured data is denoted by measured hysteresis loss P_HE. The measured hysteresis loss P_HE is not limited to those actually measured, and may be calculated from the measured magnetic susceptibility μE.

### [Step S305]

Next, the processing proceeds to Step S305, in which the data assimilation unit 232 executes data assimilation on the first damping constant α1 based on the said information related to the power loss and the estimated data obtained in Steps S100 and S200. Thereby, the data assimilation unit 232 calculates the second damping constant α2 that is the first damping constant on which the data assimilation is executed. The processing in Step S305 for executing the data assimilation on the damping constant α can be considered as one of the fourth data assimilation processing in the present embodiment.

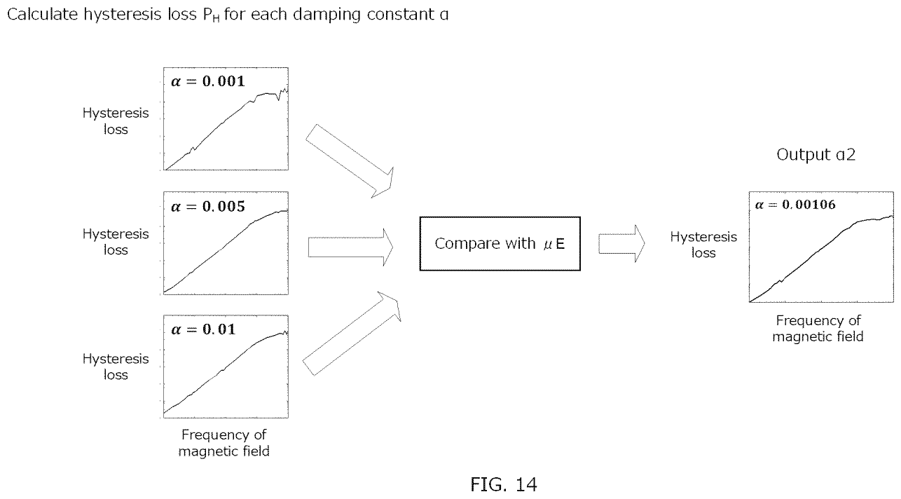

Hereinafter, an example of the method for executing the data assimilation on the first damping constant α1 in Step S305 will be explained. FIG. 14 is a flowchart that represents details of the processing in Step S305.

Firstly, in Step S305, the data assimilation unit 232 sets a plurality of the damping constants α to be used for micromagnetic simulation. In detail, the data assimilation unit 232 sets the damping constant α to be used for the micromagnetic simulation, based on the first damping constant α1. A range of the damping constant α is arbitrary, but is preferably set to include the first damping constant α1. In FIG. 14, as an example, three values of 0.001, 0.005 and 0.01 are set as the damping constants α.

Subsequently, the data assimilation unit 232 executes the micromagnetic simulation on each of the set damping constant α by using the latest estimated data (the temperature dependence M2 of the second spontaneous magnetization, the second estimated magnetic exchange coefficient Jij2, the temperature dependence K2 of the second estimated magnetic anisotropy energy and the like) and the like. Thereby, the complex magnetic susceptibility μ for each of the set damping constant α can be obtained. At this time, the data assimilation unit 232 executes the said micromagnetic simulation at plural temperatures T and frequencies of the magnetic field H, thereby obtaining a temperature and magnetic field dependence of the complex magnetic susceptibility μ for each of the set damping constant α.

The specific aspect of the micromagnetic simulation is arbitrary. The data assimilation unit 232 may evaluate a magnitude of the effective magnetic field H_eff and a value of a gyromagnetic constant γ by incorporating influence of adjacent sites on the target site as an effect of the field, based on, for example, information related to a structure of the material, the temperature dependence of the spontaneous magnetization M included in the estimated data, the temperature dependence of the magnetic anisotropy energy K, the temperature dependence of the exchange stiffness constant A and the like. The adjacent site preferably includes at least a nearest neighbor site, but is not limited to the nearest neighbor site, and may include a next nearest neighbor site or a site having a distance from the target site that is longer than that of the next nearest neighbor site.

Next, the data assimilation unit 232 calculates frequency dependence of the hysteresis loss P_H by using the complex magnetic susceptibility u, which is obtained for each damping constant α by the micromagnetic simulation.

Next, the data assimilation unit 232 compares the hysteresis loss P_H for each calculated damping constant α with the measured hysteresis loss P_HE, and calculates the damping constant α that reproduces the measured hysteresis loss P_HE as the second damping constant α2. A method for specifying the second damping constant α2 is arbitrary, but, for example, a difference between: a weighted average of the hysteresis loss P_H for each of the plural calculated damping constants α; and the measured hysteresis loss P_HE is minimized by the least squares method or the like, so that the second damping constant α2 is calculated from a coefficient included in the said weighted average. Thereby, the data assimilation is executed on the damping constant α. In the present embodiment, α2 is 0.00106. Thereafter, the processing proceeds to Step S306.

Incidentally, when the measured data does not include the information related to the power loss (when the judgment result of Step S304 is negative), the processing in Step S305 is omitted and proceeds to Step S306.

### [Step S306]

In Step S306, the output unit 234 outputs the various physical property values, which are obtained by the above-mentioned data assimilation processing and the like, as the latest estimated data. The output unit 234 outputs the second estimated data for the physical property values of which the second estimated data is obtained, and outputs the first estimated data for the physical property values of which the second estimated data is not obtained. The said estimated data includes, for example, the second estimated magnetic exchange coefficient Jij2, the temperature dependence M2 of the second spontaneous magnetization, the second damping constant α2 and the like. The output estimated data can be used for predetermined simulation such as the micromagnetic simulation and the like. Then, the processing in Step S300 is completed.

By executing the information processing as described above, the second estimated data, which provides less discrepancy with the experimental fact than that of the first estimated data, and the simulation result with higher accuracy based on the said second estimated data can be obtained.

### 4. Others

The above-described aspects of the information processing are just examples, and the present disclosure is not limited to those.

As shown in FIG. 10, a condition for the data assimilation unit 232 to execute the third data assimilation processing (in detail, the processing in Step S202) is not limited to that the difference between the first estimated magnetic exchange coefficient Jij1 and the second estimated magnetic exchange coefficient Jij2 is the first coupling threshold value or more. For example, when a difference between first estimated data and second estimated data of an arbitrary physical property that responds to the change in the coupling coefficient, such as the difference between the temperature dependence M1 of the first spontaneous magnetization and the temperature dependence M2 of the second spontaneous magnetization, is a predetermined value or more, the data assimilation unit 232 may execute the third data assimilation processing.

In Step S201, for example, the processor 23 may judge whether the difference between the temperature dependence of the order parameter included in the first estimated data (temperature dependence M1 of first estimated spontaneous magnetization) and the temperature dependence of the order parameter on which the first data assimilation processing is executed (temperature dependence M2 of second estimated spontaneous magnetization) is a first variable threshold value or more, or not. A format of the difference between the above-described temperature dependence is arbitrary, and may be subtraction, an amount of change, a rate of change, a ratio or the like. The first variable threshold value can be set arbitrarily according to accuracy required for the second estimated data.

When the difference between the temperature dependence M1 of the first estimated spontaneous magnetization and the temperature dependence M2 of the second estimated spontaneous magnetization is the first variable threshold value or more (that is, when the above-described judgment result is positive), the processing proceeds to Step S202, in which the data assimilation unit 232 executes the data assimilation on the temperature dependence K1 of the first estimated magnetic anisotropy energy based on at least one value of the second estimated data.

Incidentally, when the difference between the temperature dependence M1 of first estimated spontaneous magnetization and the temperature dependence M2 of second estimated spontaneous magnetization is less than the first variable threshold value (that is, when the above-described judgment result is negative), the processing in Step S202 is omitted and proceeds to Step S203.

Similarly, as shown in FIG. 13, a condition for the processor 23 to execute the processing in Step S302 is not limited to that the difference between the first estimated magnetic exchange coefficient Jij1 and the second estimated magnetic exchange coefficient Jij2 is the second coupling threshold value or more. For example, when a difference between first estimated data and second estimated data of an arbitrary physical property that responds to the change in the coupling coefficient, such as the difference between the temperature dependence M1 of the first spontaneous magnetization and the temperature dependence M2 of the second spontaneous magnetization, is a predetermined value or more, the processor 23 may execute the physical property simulation based on the second estimated data in Step S302 and output the first damping constant α1 by using the output unit 234.

In Step S201, for example, the processor 23 may judge whether the difference between the temperature dependence M1 of the first estimated spontaneous magnetization and the temperature dependence M2 of the second estimated spontaneous magnetization is a second variable threshold value or more, or not. The second variable threshold value can be set appropriately according to the required accuracy and calculation resource.

When the difference between the temperature dependence M1 of the first estimated spontaneous magnetization and the temperature dependence M2 of the second estimated spontaneous magnetization is the second variable threshold value or more, the processing proceeds to Step S302, in which the processor 23 executes the physical property simulation based on the second estimated data. As a result, the output unit 234 outputs the first damping constant α1.

On the other hand, when the difference between the temperature dependence M1 of the first spontaneous magnetization and the temperature dependence M2 of the second spontaneous magnetization is less than the second variable threshold value, the processing proceeds to Step S303.

The data assimilation processing in Step S3 does not required to include all of: the first data assimilation processing and the second data assimilation processing in Step S100; the third data assimilation processing in Step S200; and the fourth data assimilation processing in Step S300. For example, the data assimilation processing in Step S3 may include only the first data assimilation processing in Step S100. Also, each of the data assimilation processing may be executed independently.

The ferroic order phase to which the said information processing is applied is not limited to the ferromagnetic phase. For example, the ferroic order phase to which the said information processing is applied is an arbitrary phase, such as a ferroelectric phase, a ferroelastic phase, a ferrotoroidal phase and the like. Further, the said information processing can also be applied to an arbitrary long-range order within the material as the ferroic order. As the long-range order, an antiferromagnetic phase, a weak ferromagnetic phase, a canted antiferromagnetic phase, a helimagnetic phase, a skyrmion phase, a charge ordered phase and the like can be exemplified.

For example, if the ferroic order phase is a ferroelectric phase, the order parameter can be represented with spontaneous polarization and vibration modes of atoms. The coupling coefficient, which indicates the magnitude of the interaction, can include contribution by, for example, Coulomb interaction, overlap integral of electron orbits, spin-orbit interaction and the like. The saturation value represents a saturation value of the spontaneous polarization. The phase transition temperature represents the Curie temperature which indicates phase transition from the ferroelectric phase to a paraelectric phase. The field conjugate to the ferroic order phase is an electric field. The susceptibility represents electric susceptibility (in particular, complex electric susceptibility). Relations between the above-described physical property values can be obtained by a time-dependent Landau-Lifshitz equation with respect to polarization, a linear response theory, a Landau's phenomenological theory based on symmetry, a molecular field theory or the like. The same applies to the other ferroic order phases.

If the measured data has been input into the user terminal 3 in Step S2, the information processing apparatus 2 may acquire the measured data, which is input into the user terminal 3, from the user terminal 3. Also, in the case where the information processing apparatus 2 itself functions as a measurement device, the acquisition unit 231 may acquire the measurement result obtained by the information processing apparatus 2 as the measured data.

The information processing apparatus 2 may be on-premises or in-cloud. The in-cloud information processing apparatus 2 may provide the above-described functions and processing in a form of, for example, Saas (Software as a Service) or cloud computing.

In the above-described embodiment, the information processing apparatus 2 executes various storage and control operations, but a plural external devices may be used instead of the information processing apparatus 2. In other words, various information and programs may be divided to be stored into the plurality of the external devices by using blockchain technology or the like.

The aspect of the present embodiment is not limited to the information processing system 1, and may be an information processing method or an information processing program. The information processing method includes each of the steps of the information processing system 1. The information processing program allows at least one computer to execute each of the steps of the information processing system 1.

The above-described information processing system 1 and the like may be provided in each of following aspects.

(1) An information processing system, comprising at least one processor configured to execute a program to perform each step of: an acquisition step of acquiring first estimated data related to a physical property of a material, which is calculated by predetermined physical property simulation based on a model of the material having a ferroic order phase, and measured data obtained by measurement of the material, wherein the first estimated data includes: temperature dependence of an order parameter in the ferroic order phase; and a coupling coefficient representing magnitude of interaction between sites of the material which contributes to formation of the ferroic order phase; a data assimilation processing step of, when the measured data includes a measured reference value, executing first data assimilation processing on the coupling coefficient by multiplying: a ratio of the measured reference value to an estimated reference value; with the coupling coefficient included in the acquired first estimated data, according to a degree which represents dependence of the estimated reference value on the coupling coefficient, wherein the measured reference value includes at least one of: a phase transition temperature representing phase transition from the ferroic order phase which is caused due to the order parameter becoming zero; and a saturation value which is a value of the order parameter corresponding to a saturated state of the ferroic order phase at an absolute zero point, and the estimated reference value is a value corresponding to the measured reference value among the phase transition temperature and the saturation value which are included in the acquired first estimated data; and an output step of outputting the coupling coefficient, on which the first data assimilation processing is executed, as second estimated data.

According to this configuration, the first estimated data includes information that is related to ideal physical properties of the material as a target to be measured. The measured data includes information, which is intrinsic to the material as the target to be measured, such as quality of the material and measurement conditions. Therefore, the second estimated data, which is calculated from the first estimated data and the measured data, includes the information intrinsic to the material that is reflected on the ideal physical properties of the material. Herein, the coupling coefficient represents strength of the interaction between the sites forming the ferroic order phase. Therefore, the coupling coefficient is an important factor for specifying characteristics of the ferroic order phase such as a physical property value generated by the ferroic order phase and a spatial property for domain formation and the like. Accordingly, by enhancing accuracy of the coupling coefficient by the above-described data assimilation, a deviation between a result of the physical property simulation on the ferroic order phase and a measurement result of the material can be suppressed.

(2) The information processing system according to (1), wherein the data assimilation processing step further includes executing second data assimilation processing on the temperature dependence of the order parameter included in the acquired first estimated data by multiplying the acquired temperature dependence of the order parameter based on the ratio, and the output step further includes outputting the temperature dependence of the order parameter, on which the second data assimilation processing is executed, as the second estimated data.

According to such a configuration, the temperature dependence of the order parameter, which is more in accordance with the experimental result than that of the first estimated data, can be obtained. Herein, the magnitude of the order parameter implies temperature dependence of energy required for the phase transition from the ferroic order phase. Therefore, reliability of a temperature design of the device utilizing the phase transition of the ferroic order phase can be enhanced.

(3) The information processing system according to (2), wherein the physical property simulation includes: first-principles calculation for outputting the first estimated data at the absolute zero point based on the model of the material; and finite temperature calculation for outputting the first estimated data at a finite temperature based on the first estimated data at the absolute zero point, wherein the first estimated data at the absolute zero point includes the coupling coefficient, and the second data assimilation processing includes executing the finite temperature calculation again by using the coupling coefficient on which the first data assimilation processing is executed.

According to such a configuration, since the information related to the physical property at the finite temperature is calculated using the parameter that is in accordance with the measurement result, the estimated data, which is more reliable and more in accordance with the experimental fact than that obtained by executing finite temperature calculation just by using the result of the first-principles calculation, can be obtained.

(4) The information processing system according to (2) or (3), wherein the data assimilation processing step further includes, when the measured data includes at least one value of the order parameter in the material at a finite temperature other than the phase transition temperature, correcting the temperature dependence of the order parameter included in the estimated data based on the value of the order parameter in the material at the finite temperature.

According to such a configuration, the reliability of the temperature dependence of the order parameter at the finite temperature between the absolute zero point and the phase transition point can be enhanced.

(5) The information processing system according to any one of (2) to (4), the first estimated data further includes temperature dependence of anisotropy energy representing magnitude of anisotropy of the order parameter, the data assimilation step further includes, when a difference between: the coupling coefficient included in the first estimated data; and the coupling coefficient on which the data assimilation processing is executed is a first coupling threshold value or more, or when a difference between: the temperature dependence of the order parameter included in the first estimated data; and the temperature dependence of the order parameter on which the data assimilation processing is executed is a first variable threshold value or more, executing third data assimilation processing on the temperature dependence of the anisotropy energy included in the first estimated data based on the second estimated data, and the output step further includes outputting the temperature dependence of the anisotropy energy, on which the third data assimilation processing is executed, as the second estimated data.

According to such a configuration, reliability of estimation accuracy of a direction of the order parameter is enhanced.

(6) The information processing system according to (5), wherein the data assimilation processing step includes, when the measured data includes the temperature dependence of the anisotropy energy, correcting the temperature dependence of the anisotropy energy included in the estimated data based on the temperature dependence of the anisotropy energy.

According to such a configuration, the reliability of the estimation accuracy of the direction of the order parameter is further enhanced.

(7) The information processing system according to any one of (2) to (6), wherein the output step includes, when a difference between: the coupling coefficient included in the first estimated data; and the coupling coefficient included in the second estimated data is a second coupling threshold value or more, or when a difference between: the temperature dependence of the order parameter included in the first estimated data; and the temperature dependence of the order parameter included in the second estimated data is a second variable threshold value or more, outputting temperature dependence of a first damping constant by executing the physical property simulation based on the second estimated data, wherein the damping constant represents a degree of microscopic damping of the order parameter at the site.

According to such a configuration, the damping constant is one of factors that determine a relaxation process of the ferroic order phase. Therefore, due to the obtainment of the damping constant which is in accordance with the experimental fact, the reliability of the simulation on the physical property of the ferroic order phase by using the damping constant can be enhanced.

(8) The information processing system according to (7), wherein the data assimilation processing step includes, when the measured data includes information related to power loss in the material which is caused by application of a field conjugate to the order parameter, executing fourth data assimilation processing on the first damping constant based on the information related to the power loss and the estimated data, and the output step includes outputting a second damping constant that is the first damping constant on which the fourth data assimilation processing is executed.

According to such a configuration, since the damping constant is estimated based on the plural experimental facts, estimation accuracy of the damping constant is enhanced. Therefore, the reliability of the simulation on the physical property of the ferroic order phase by using the damping constant can be further enhanced.

(9) The information processing system according to any one of (1) to (8), wherein the ferroic order phase is a ferromagnetic phase, the order parameter is spontaneous magnetization of the material, the coupling coefficient is a magnetic coupling coefficient between the sites, the phase transition temperature is a Curie temperature that corresponds to phase transition from the ferromagnetic phase to a paramagnetic phase, and the saturation value is saturation magnetization of the material.

According to such a configuration, the information intrinsic to the material, which is included in the measured data, is reflected on the estimated data of the various physical property values related particularly to the ferromagnetism. Thus, for example, convenience of designing a device that utilizes the ferromagnetic property can be enhanced.

(10) An information processing method, comprising each of the steps of the information processing system according to any one of claims 1 to 9.

(11) An information processing program, configured to allow at least one computer to execute each of the steps of the information processing system according to any one of claims 1 to 9.

Needless to say, the present disclosure is not limited to the above description.

Finally, various embodiments of the invention have been described, which are presented as examples and are not intended to limit the scope of the invention. The novel embodiments can be implemented in various other forms, and various omissions, substitutions, and modifications can be made to the extent that they do not depart from the gist of the invention. The embodiment and variations thereof are included in the scope or gist of the invention and within the scope of the invention and its equivalents described in the claims.

1. An information processing system, comprising at least one processor configured to execute a program to perform each step of:
an acquisition step of acquiring first estimated data related to a physical property of a material, which is calculated by predetermined physical property simulation based on a model of the material having a ferroic order phase, and measured data obtained by measurement of the material, wherein
the first estimated data includes: temperature dependence of an order parameter in the ferroic order phase; and a coupling coefficient representing magnitude of interaction between sites of the material which contributes to formation of the ferroic order phase;
a data assimilation processing step of, when the measured data includes a measured reference value, executing first data assimilation processing on the coupling coefficient by multiplying: a ratio of the measured reference value to an estimated reference value; with the coupling coefficient included in the acquired first estimated data, according to a degree which represents dependence of the estimated reference value on the coupling coefficient, wherein
the measured reference value includes at least one of: a phase transition temperature representing phase transition from the ferroic order phase which is caused due to the order parameter becoming zero; and a saturation value which is a value of the order parameter corresponding to a saturated state of the ferroic order phase at an absolute zero point, and
the estimated reference value is a value corresponding to the measured reference value among the phase transition temperature and the saturation value which are included in the acquired first estimated data; and
an output step of outputting the coupling coefficient, on which the first data assimilation processing is executed, as second estimated data.

2. The information processing system according to claim 1, wherein
the data assimilation processing step further includes executing second data assimilation processing on the temperature dependence of the order parameter included in the acquired first estimated data by multiplying the acquired temperature dependence of the order parameter based on the ratio, and
the output step further includes outputting the temperature dependence of the order parameter, on which the second data assimilation processing is executed, as the second estimated data.

3. The information processing system according to claim 2, wherein
the physical property simulation includes: first-principles calculation for outputting the first estimated data at the absolute zero point based on the model of the material; and finite temperature calculation for outputting the first estimated data at a finite temperature based on the first estimated data at the absolute zero point, wherein
the first estimated data at the absolute zero point includes the coupling coefficient, and
the second data assimilation processing includes executing the finite temperature calculation again by using the coupling coefficient on which the first data assimilation processing is executed.

4. The information processing system according to claim 2, wherein
the data assimilation processing step further includes, when the measured data includes at least one value of the order parameter in the material at a finite temperature other than the phase transition temperature, correcting the temperature dependence of the order parameter included in the estimated data based on the value of the order parameter in the material at the finite temperature.

5. The information processing system according to claim 2,
the first estimated data further includes temperature dependence of anisotropy energy representing magnitude of anisotropy of the order parameter,
the data assimilation step further includes, when a difference between: the coupling coefficient included in the first estimated data; and the coupling coefficient on which the data assimilation processing is executed is a first coupling threshold value or more, or when a difference between: the temperature dependence of the order parameter included in the first estimated data; and the temperature dependence of the order parameter on which the data assimilation processing is executed is a first variable threshold value or more, executing third data assimilation processing on the temperature dependence of the anisotropy energy included in the first estimated data based on the second estimated data, and
the output step further includes outputting the temperature dependence of the anisotropy energy, on which the third data assimilation processing is executed, as the second estimated data.

6. The information processing system according to claim 5, wherein
the data assimilation processing step includes,
when the measured data includes the temperature dependence of the anisotropy energy,
correcting the temperature dependence of the anisotropy energy included in the estimated data based on the temperature dependence of the anisotropy energy.

7. The information processing system according to claim 2, wherein
the output step includes, when a difference between: the coupling coefficient included in the first estimated data; and the coupling coefficient included in the second estimated data is a second coupling threshold value or more, or when a difference between: the temperature dependence of the order parameter included in the first estimated data; and the temperature dependence of the order parameter included in the second estimated data is a second variable threshold value or more, outputting temperature dependence of a first damping constant by executing the physical property simulation based on the second estimated data, wherein
the damping constant represents a degree of microscopic damping of the order parameter at the site.

8. The information processing system according to claim 7, wherein
the data assimilation processing step includes, when the measured data includes information related to power loss in the material which is caused by application of a field conjugate to the order parameter, executing fourth data assimilation processing on the first damping constant based on the information related to the power loss and the estimated data, and
the output step includes outputting a second damping constant that is the first damping constant on which the fourth data assimilation processing is executed.

9. The information processing system according to claim 1, wherein
the ferroic order phase is a ferromagnetic phase,
the order parameter is spontaneous magnetization of the material,
the coupling coefficient is a magnetic coupling coefficient between the sites,
the phase transition temperature is a Curie temperature that corresponds to phase transition from the ferromagnetic phase to a paramagnetic phase, and
the saturation value is saturation magnetization of the material.

10. An information processing method, comprising each of the steps of the information processing system according to claim 1.

11. A non-transitory computer-readable memory medium storing an information processing program configured to allow at least one computer to execute each of the steps of the information processing system according to claim 1.
